(19) United States
(12) Patent Application Publication
(10) Pub. No.: US 2008/0145438 A1
(43) Pub. Date: Jun. 19, 2008

(54) Micro- and nano-particulate drugs and methods of making thereof

(76) Inventor: Beuford Arlie Bogue, Broad Run, VA (US)

Correspondence Address: THORPE NORTH & WESTERN, LLP., P.O. Box 1219, SANDY, UT, 84091-1219, US

(21) Appl. No.: 11/823,476

(22) Filed: Jun. 26, 2007

Related U.S. Application Data
(63) Continuation of application No. 10/022,799, filed on Dec. 20, 2001, now abandoned.

(51) Int. Cl. A61K 9/51; A61K 9/50; A61P 43/00; A61K 31/522

(52) U.S. Cl. 424/490; 514/263.34

(57) ABSTRACT

A micro and nano-particulate drug comprising a drug substance and a surfactant in which the drug and surfactant form a eutectic mixture. The matrix formed between the drug substance and the surfactant has a melting point less than the decomposition temperature of the drug substance and thus provides the advantages of reduced irritation due to the melting process without the prior art problem of decomposition of the drug substance. In one embodiment, crystals are formed while the mixture is cooled at room temperature under high shear conditions. In a second embodiment, a flowable material may be formed which also contains the drug and that may be incorporated into a pharmaceutical delivery system is also disclosed. Methods of preparing the micro and nano-particulate drug crystals and non-crystalline substance are also contemplated in the inventive subject matter.

### PRIORITY CLAIM

This application is a continuation of U.S. patent application Ser. No. 10/022,799, filed Dec. 20, 2001, which is hereby incorporated herein by reference in its entirety.

### FIELD OF THE INVENTION

The present invention relates to micro- and nano-particulate drug crystals having improved solubility, absorption and wettability characteristics. The invention also relates to a surfactant-drug matrix wherein the melting point of the matrix is less than a decomposition temperature of the drug. The present invention further relates to a method of preparing the micro- and nano-particulate drug crystals wherein the eutectic mixture is subject to high shear during a cooling step.

### DESCRIPTION OF THE RELATED ART

Over the years, compositions and methods have been developed to achieve improved delivery of a therapeutically effective amount of a drug. In particular, compositions and methods relating to enhanced solubility, absorption and wettability characteristics of a drug resulting in a desired dissolution rate in vivo have been sought.

One of the reasons for the increased focus in this field of art is the poor solubility characteristics of new pharmaceuticals. Many newly developed drugs possess poor absorption profiles and unfavorable dissolution characteristics. A cursory review of pharmacokinetic characteristics of recently developed drugs suggests that more than 40 percent of the drug substances have aqueous solubilities below 1 mg/ml, and that 32 percent have an aqueous solubility below 0.1 mg/ml. The low solubility of drugs in water and in organic solvents translates into a lowered ability to deliver the drug to an animal in need thereof.

For example, potential absorption problems may occur in the oral route of administration unless the substance has an aqueous solubility above 10 mg/ml over the pH-range 1-7. Pharmacological testing is also hampered because following oral or intramuscular administration, it is not possible to test the bioavailability of the drug due to low solubility.

Accordingly, implementation of absorption enhancing methods is a major field in the formulation of drug dosage forms.

One known method to overcome low dissolution rate of a drug is by reducing particle size thereby causing the surface area available for interaction with the fluids to be significantly increased. For drugs, where absorption is limited by dissolution rate, particle size reduction clearly represents a viable means for increasing bioavailability. In particular, the dissolution rate increases as the particle surface area increases in accordance with the Noyes-Whitney law. As a result, the rate of flooding of active compounds increases, and the maximum plasma level is reached faster (e.g. oral or i.v. administration of a micro- and nano-particulate drug crystals). Aqueous solubility of drug substances is also improved by particle size reduction.

One advantage of reducing particle size is that intravenous administration of insoluble or sparingly soluble active drugs can be accomplished. Moreover, sparingly soluble active compound can be injected without blockade by blood capillaries.

Another advantage is a reduction in the injection volume of drugs. For example, if the water-solubility is low, a relatively large volume is administered. Alternatively, micro- and nano-particulate drug crystals can be dispersed in a saturated solution of the active compound thereby reducing the volume of the injection.

Small particle drugs can also be employed for controlled drug delivery. For example, after oral administration, oral immunization could take place via the M cells in the gastrointestinal tract, and selective concentration in the absorption windows of the gastrointestinal tract could be achieved via bioadhesives.

Another use is drug targeting. After intravenous injection, it is well known in the art that particles accumulate specifically in certain organs, e.g. liver, spleen or bone marrow, as a function of their surface properties. Therefore, after administration, accumulation in targeted organs can be achieved. Targeted accumulation of the active compound at the site of action reduces side effects and increases therapeutic efficiency.

Accordingly, many techniques have been developed to reduce the particle size of a drug. The majority relate to various milling techniques wherein the drug is comminuted by dry grinding techniques and subsequent fractionation. However, the disadvantage is the loss of the active compound during the milling process. Sometimes the milling process may waste up to more than 90% of the active thereby greatly reducing cost effectiveness.

One technique attempts to circumvent this loss by providing for a high molecular weight polymer which provides a higher processing temperature and a longer period for the manipulation of a resin and drug in a mill or other processing machine. These conditions increase the amount of drug that can be dissolved in the resin without degrading the resin, and the relative rigidity of the resin can assist in its grinding to form granular particles or powders.

Surfactant-stabilized microparticles have also been patented (U.S. Pat. No. 5,246,707), it also being possible for these additionally to comprise iron particles within the microparticles in order to allow location of the particles via magnetic fields.

Preparation of micro- and nano-particulate drug crystals by wet grinding has been patented by Motoyanna et al. as a process (U.S. Pat. No. 4,540,602) and wet grinding with a pearl mill has been patented by Liversidge et al. (U.S. Pat. No. 5,145,684). A further reduction in the particle size in such mills is possible if the viscosity of the dispersion medium is increased, but the speed of rotation must remain constant.

However, the above outlined milling techniques have the disadvantages of not being amenable to industries of scale and result in relatively large particles. Moreover, the techniques are only applicable to certain classes of molecules and do not ensure homogenous results.

One technique to overcome these disadvantages is to produce the suspensions by precipitation. EP 0 275 79 discloses a preparation of a liquid phase consisting of a solution of the drug added to a second liquid phase consisting of a non-solvent or a mixture of non-solvents of the substance to which one or more surfactants may be added, the non-solvent or the mixture of non-solvents for the substance being miscible in all proportions with the solvent or mixture of solvents for the substance and wherein the both phases are mixed with moderate agitation so as to produce a colloidal suspension of particles of the substance.

The disadvantage is that the technique is limited to substances sufficiently soluble in water or a given solvent.

U.S. Pat. No. 5,858,410 (“Muller et al.”) attempts to sidestep the problem of insoluble or sparingly soluble drugs by avoiding a precipitation technique. In particular, Muller et al. discloses that drugs having low solubility can have an increased dissolution rate by using an ultrasonic probe, a ball mill or a pearl mill, wherein the drug is comminuted by using cavitation or shearing and impact forces with introduction of a high amount of energy without prior conversion into a melt.

However, a disadvantage of this milling technique is that the residual content of solvents in the product can only be removed with great difficulty which delays crystallization and many times produces a high proportion of large particles.

Moreover, recent investigations directly contravene Muller et al. by suggesting that crystals derived from a melt give rise to significant advantages over crystals derived from milling. In particular, crystals co-precipitated out of a melt are significantly less irritating than the solid dispersions created by milling. (“Comparative Evaluation of Controlled-Release Solid Oral Dosage Forms Prepared with Solid Dispersions and Coprecipitates”, Khan, Shojael, Karnachi, Keddy, Pharmaceutical Technology (May 1999)). In vivo ulcerogenicity data clearly indicates that drugs coprecipitated from a melt produces less gastric irritation. Id. at 16.

Finally, no known methods overcome the difficulty of melting a drug without decomposing the drug or for providing a drug surfactant matrix wherein the surfactant coats the drug.

Accordingly, one aspect of the invention is to provide micro- and nano-particulate drug crystals having improved solubility, absorption and wettability characteristics.

Another aspect of the invention is to provide for a surfactant-drug matrix of drug and surfactant wherein the melting point of the matrix is less than a decomposition temperature of the drug.

Yet another aspect of the invention is to provide for a method of making micro- and nano-particulate drug crystals having improved solubility, absorption and wettability characteristics.

Still yet another aspect of the invention is to provide for a method of making micro- and nano-particulate drug crystals from insoluble or sparingly soluble drugs.

Further still yet another aspect of the invention is to provide for a method of making micro- and nano-particulate drug crystals comprising a eutectic melt.

Another aspect of the invention is to provide for a method of making micro- and nano-particulate drug crystals wherein the crystals are less irritating to the animal in need thereof.

Yet another aspect of the invention is to provide for a method of making micro- and nano-particulate drug crystals wherein the crystals have desirable release characteristics.

Still yet another aspect of the invention is to provide for a method of making micro- and nano-particulate drug crystals wherein the crystals can be used in a variety of dosage forms.

These and other aspects of the invention will be apparent for the detailed description and the claims.

### SUMMARY OF THE INVENTION

Applicant has unexpectedly produced a micro or nano-particulate drug composition comprising: a drug substance; and a surfactant; wherein said surfactant and said drug substance form a surfactant-drug substance matrix at a temperature above said matrix's melting temperature and wherein said surfactant is miscible with said drug substance and does not chemically bond with said drug substance; and wherein said drug substance and said surfactant form a non-crystalline substance or micro or nano-sized crystals containing said drug while being cooled to room temperature under a shearing force.

A further preferred embodiment is drawn to a method of making a micro and nano-particulate drug, the steps comprising: providing a drug substance-surfactant mixture; melting the mixture at a temperature above said mixture's melting temperature; and cooling the mixture under high shear to approximately room temperature, wherein a non-crystalline substance forms or crystals precipitate coated with said surfactant during crystallization.

The present inventive subject matter relates to micro- and nano-particulate drug crystals having improved solubility, absorption and wettability characteristics. The invention also relates to surfactant-drug matrix wherein the melting point of the matrix is less than a decomposition temperature of the drug. The present invention further relates to a method of preparing the micro- and nano-particulate drug crystals wherein the eutectic mixture is subject to high shear during a cooling step.

### BRIEF DESCRIPTION OF THE DRAWINGS

The invention may be better understood by reference to the drawings wherein:

FIG. 1 is a phase diagram of a surfactant/drug eutectic mixture.

FIG. 2 is a graphical representation of solubility characteristics plotted as solubility vs. decrease in particle size.

FIG. 3 is a graphical representation of wettability characteristics plotted as percent dissolved vs. time.

FIG. 4 is a graphical representation of dimenhydrinate dissolution enhancement with the drug dissolved plotted as percent dissolved vs. time.

FIG. 5 is a graphical representation of the effects of intensive nucleation on matrix solubility with the drug dissolved plotted as percent vs. time.

FIG. 6 is a graphical representation of the effect of drug loading on matrix solubility with the drug dissolved plotted as percent dissolved vs. time.

### DETAILED DESCRIPTION OF THE INVENTION

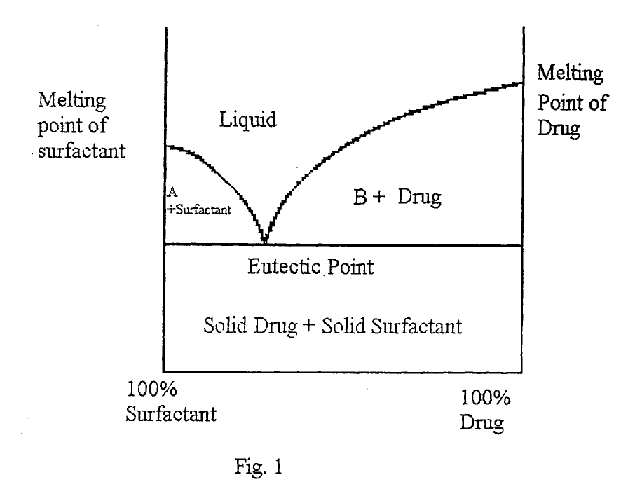

As used herein the term “phase diagram” indicates a graphical representation of limiting conditions of temperature and pressure or solubility and temperature under which two phases are in equilibrium with one another as shown in FIG. 1.

As used herein the term “two-component phase diagram” indicates a graphical representation of two component phase regions Surfactant+liquid, Drug+liquid and a solid phase (Surfactant+Drug composition) under which those components are stable as shown in FIG. 1.

As used herein the term “eutectic” indicates a mixture of two or more substances which liquefies at the lowest temperature of all such mixtures, where a liquid mixture of two miscible substances is cooled, one component will begin to separate in its solid form.

As used herein, “particle size” refers to a number average particle size as measured by conventional particle size measuring techniques well known to those skilled in the art, such as sedimentation field flow fractionation, photon correlation spectroscopy, or disk centrifugation.

As used herein, “room temperature” refers to a temperature at or around 40° C. or below.

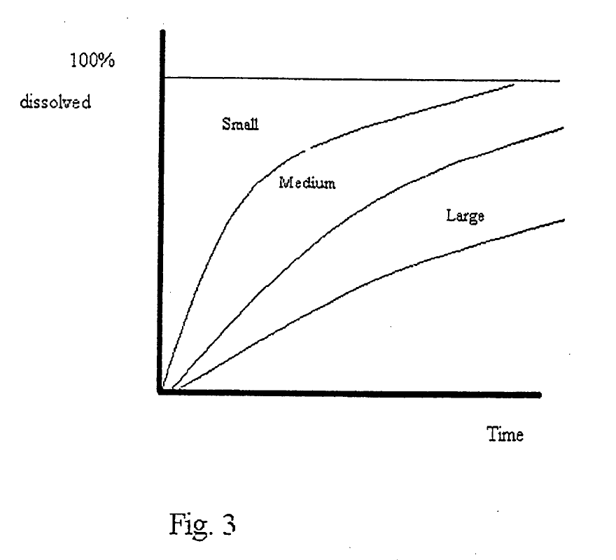

As used herein, “wettability” refers to the ability of a substance to be dissolved in solution in relation to time vs. particle size as shown in FIG. 3.

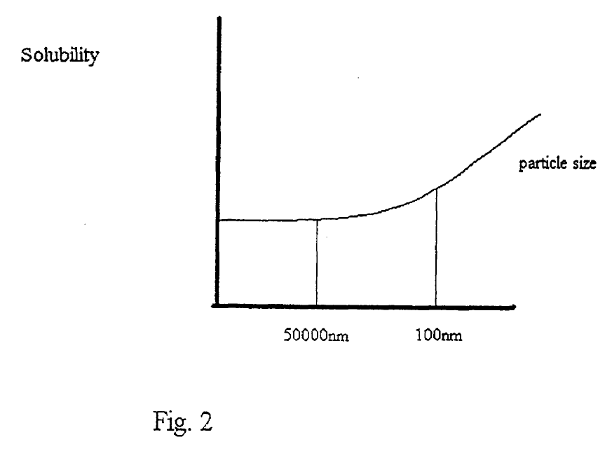

As used herein, “solubility” refers to the amount of a substance that can be dissolved in a given amount of solvent as shown in FIG. 2.

As used herein, “absorption” refers to the process of drug movement from the site of administration toward the systemic circulation.

The present invention relates to a micro- and nano-particulate drug crystals having improved solubility, absorption and wettability characteristics. The invention also relates to surfactant-drug matrix of drug and surfactant wherein the melting point of the matrix is less than a decomposition temperature of the drug. The present invention further relates to a method of preparing the micro- and nano-particulate drug crystals wherein the eutectic matrix is subject to high shear during a cooling step.

Typically, the micro- and nano-particulate crystals are in the nanometer to micrometer range denoted by (“nm”) and (“.mu.”), respectively.

The invention can be practiced with a wide variety of drug substances. The drug substance preferably is present in an essentially pure form. The drug substance can be poorly or sparingly soluble and dispersible in at least one liquid medium. By “poorly or sparingly soluble” it is meant that the drug substance has a solubility in a liquid dispersion medium of less than about 100 mg/ml, and preferably of less than about 1 mg/ml. A preferred liquid dispersion medium is water. However, the invention can be practiced with other liquid media in which a drug substance is poorly soluble and dispersible including, for example, aqueous salt solutions, safflower oil and solvents such as ethanol, t-butanol, hexane and glycol. The pH of the aqueous dispersion media can be adjusted by techniques known in the art.

Suitable drug substances can be selected from a variety of known classes of drugs including, for example, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators and xanthines. Preferred drug substances include those intended for oral administration and intravenous administration. A description of these classes of drugs and a listing of species within each class can be found in Martindale, The Extra Pharmacopoeia, Twenty-ninth Edition, The Pharmaceutical Press, London, 1989, the disclosure of which is hereby incorporated by reference in its entirety. The drug substances are commercially available and/or can be prepared by techniques known in the art.

The particles of this invention contain a discrete phase of a drug substance as described above having a surfactant adsorbed on the surface thereof. Useful surfactants are believed to include those which physically adhere to the surface of the drug substance but do not chemically bond to the drug.

Suitable surfactants can preferably be selected from known organic and inorganic pharmaceutical excipients. Such excipients include various polymers, low molecular weight oligomers, natural products and surfactants. Preferred surfactants include nonionic and anionic surfactants. Representative examples of excipients include gelatin, casein, lecithin (phosphatides), gum acacia, cholesterol, tragacanth, stearic acid, benzalkonium chloride, calcium stearate, glyceryl monostearate, cetostearl alcohol, cetomacrogol emulsifying wax, sorbitan esters, polyoxyethylene alkyl ethers, e.g., macrogol ethers such as cetomacrogol 1000, polyoxyethylene castor oil derivatives, polyoxyethylene sorbitan fatty acid esters, e.g., the commercially available Tweens, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, and polyvinylpyrrolidone (PVP). Most of these excipients are described in detail in the Handbook of Pharmaceutical Excipients, published jointly by the American Pharmaceutical Association and The Pharmaceutical Society of Great Britain, the Pharmaceutical Press, 1986, the disclosure of which is hereby incorporated by reference in its entirety. The surface modifiers are commercially available and/or can be prepared by techniques known in the art.

Particularly preferred surfactants include polyvinyl alcohol (“PEG”), polyvinyl pyrrolidone, Pluronic F68 and F108, which are block copolymers of ethylene oxide and propylene oxide, Tetronic 908, which is a tetrafunctional block copolymer derived from sequential addition of ethylene oxide and propylene oxide to ethylenediamine, dextran, lecithin, Aerosol OT, which is a dioctyl ester of sodium sulfosuccinic acid, available from American Cyanamid, Duponol P, which is a sodium lauryl sulfate, available from DuPont, Triton X-200, which is an alkyl aryl polyether sulfonate, available from Rohm and Haas, Tween 80, which is a polyoxyethylene sorbitan fatty acid ester, available from ICI Specialty Chemicals, and Carbowax 3350 and 934, which are polyethylene glycols available from Union Carbide. Surface modifiers which have found to be particularly useful include polyvinylpyrrolidone, Pluronic F-68, and lecithin.

It should be noted that the surfactants do not chemically react with the drug substance or itself. Furthermore, the individually adsorbed molecules of the surfactants are essentially free of intermolecular crosslinkages.

In preferred embodiments of the present inventive subject matter, the effective average particle size of the formed drug is less than about 5 microns, and preferably less than about 400 nm. In even more preferred embodiments of the invention, the effective average particle size of the formed drug crystals is less than about 250 nm. In some embodiments of the invention, an effective average particle size of less than about 100 nm may be achieved. With reference to the effective average particle size of the drug crystals, it is preferred that at least 50% and, more preferably, at least 75% of the particles have a particle size less than the effective average, e.g., 5 microns and more preferably, 400 nm. In particularly preferred embodiments, essentially all of the particles have a size less than 5 microns and preferably less than 400 nm. In some embodiments, essentially all of the particles have a size less than 250 nm.

The micro- and nano-particulate crystals of the present invention can be made by a variety of devices which provides sufficiently high shear for shear mixing. There are a large variety of these devices available on the market readily ascertainable by one of ordinary skill in the art for the intended purpose of the present invention.

The method for making the micro- and nano-particulate crystals of the present invention comprises the steps of providing a drug surfactant mixture and melting the resulting mixture and cooling the mixture continuously under high shear to a temperature at or around room temperature.

Typically, the drug will be melted below its decomposition temperature when added to molten surfactant. This has the advantage of avoiding the decomposition temperature of the drug because a eutectic mixture generally has a melting temperature lower than the highest individual melting point of either component. Accordingly, the surfactant/drug combination liquefies at a temperature below the decomposition temperature of the drug. Nevertheless, for stability reasons, the surfactant should have a melting point above about room temperature, and preferably above about 40° C.

One way to test for the dissolution characteristic of the resultant crystals is by observing whether the molten eutectic mixture is cloudy. If the molten eutectic mixture is cloudy a paste will form. On the other hand, if the molten eutectic mixture is clear, micro- and nano-particulate crystals will form.

It should be noted that the surfactant must be miscible with the drug. Thus, in most cases, the surfactant will be an organic solvent such that the liquid phase (1) will constitute the organic phase whereas the liquid phase, and (2) will constitute the aqueous phase; but it is possible to use either two organic phases or two aqueous phases provided the conditions regarding solubility, insolubility and miscibility are met. On the other hand, the solvent must be sufficiently volatile for it to be removed if necessary. For example, in the case in which the substance is a polymer (to which a biologically active substance has or has not been added), the solvent may be chosen from among a lower alcohol (methanol, ethanol, isopropanol, etc.), a lower ketone (acetone, methyl-ethyl-ketone, etc.), a light hydrocarbon or a mixture of light hydrocarbons (hexane, petroleum ether, etc.), a chlorinated light hydrocarbon (chloroform, methylene chloride, trichloroethylene, etc.) or other common light solvents such as acetonitrile, dioxane etc.

Additionally, it will be noted that cooling under high shear must occur until the eutectic mixture is cooled to a temperature at or around room temperature. Using this process, micro- and nano-particulate drug crystals will form coated with surfactant during the crystallization process.

If cooling does not occur under high shear until the mixture is cooled to at or around room temperature, crystals will grow on the exterior of the surfactant. Surfactant with crystals on the exterior have very poor dissolution.

However, in some cases, it will be the case that the eutectic mixture will have a eutectic point above room temperature. One way to overcome this disadvantage is by micronizing the drug prior to melting. This can be done by an extruder. An example of such an extruder is a twin-screw extruder known to those generally skilled in the art. However, due to the high surface area and poor wettability characteristics, a poor product is generally formed wherein a thick paste is usually produced at a temperature at about 40° C. and almost a dry powder is produced at 55° C.

The advantage of providing for a surfactant and drug phase is controlled precipitation of the drug from the surfactant mixture. In particular, this process allows the drug to crystallize or precipitate out of the eutectic melt rather than requiring removal of the solvent.

As the surfactant/drug mixture cools, the drug component will begin to separate out of the liquid. The remaining liquid will then continuously become richer with drug, until eventually, the composition of the remaining liquid reaches a value at which both drug and surfactant begin to separate simultaneously as an intimate mixture of drug and surfactant. This composition is known as a eutectic composition and the temperature at which it solidifies is the eutectic temperature.

Without limiting the theory of the invention to any particular theory, one possible explanation is provided for the formation of the micro- and nano-particulate drug crystals of the present invention. In particular, as the eutectic mixture cools under high shear, the high shear causes the creation of multiple nucleation sites. Increased nucleation sites translate into smaller crystals. By providing high shear during the cooling process increased nucleation sites are created.

It should also be recognized by one of ordinary skill in the art that the process described herein may result in the formation of a solid product that is not crystalline and may be flowable. Such products may be conveniently processed by techniques well known in the art to form products having sizes of about 16 mesh. This enables incorporation of the non-crystalline products into various pharmaceutical delivery systems wherein the delivery systems can be independently selected from the group consisting of tablets, bi-layer tablets, capsules, gelatin capsules, caplets, lozenges, chewable lozenges, beads, powders, granules, dispersible granules, cachets, patches, particle inhalants, implants, ingestibles, injectable or infuseables.

The present invention also contemplates substances made from either the micro- and nano-particulate drug crystals or the flowable solids. The substances can be administered by any route, including without limitation, oral, buccal, sublingual, rectal, parenteral, topical, inhalational, injectable and transdermal routes. Using the present invention with any of the above routes of administration or dosage forms can be performed using well known procedures and techniques available to one of ordinary skill in the art.

The present invention also contemplates the use of pharmaceutically acceptable carriers which may be prepared from a wide range of materials. Without being limited thereto, such materials include diluents, binders and adhesives, lubricants, plasticizers, disintegrants, colorants, bulking substances, flavorings, sweeteners and miscellaneous materials such as buffers and adsorbents in order to prepare a particular medicated composition.

Binders may be selected from a wide range of materials such as hydroxypropylmethylcellulose, ethylcellulose, or other suitable cellulose derivatives, povidone, acrylic and methacrylic acid co-polymers, pharmaceutical glaze, gums, milk derivatives, such as whey, starches and derivatives, as well as other conventional binders well known to persons skilled in the art. Exemplary non-limiting solvents are water, ethanol, isopropyl alcohol, methylene chloride or mixtures and combinations thereof. Exemplary non-limiting bulking substances include sugar, lactose, gelatin, starch, and silicon dioxide.

One of ordinary skill in the art will understand that the particular theory of the invention as described herein is not limited to any single one of the above theories, or that there may be a combination of the above theories or involve theories as of yet not ascertainable and do not limit in any way to the ability to practice the invention as disclosed herein.

Compositions and methods for the preparation of such micro- and nano-particulate drug crystals will be readily apparent to those skilled in the art, in view of the present disclosure, when the present disclosure is coupled with information known in the art.

The following examples are given to illustrate the invention, but are not deemed to be limiting thereof. All percentages given throughout the specification are based upon weight unless otherwise indicated.

### EXAMPLE 1

The following example demonstrates the enhanced dissolution of dimenhydrate and the effect of drug loading on matrix solubility.

Five grams of dimenhydrinate and 5 grams of Pluronic NF F68 were combined in a beaker and heated to approximately 90° C. until a clear liquid was obtained. The material was then transferred to a mortar and pestle at room temperature and was allowed to solidify while being vigorously ground. The resulting materials, upon becoming completely solidified, was then ground to a powder and submitted for dissolution testing. The dissolution data is presented in TABLE I.

TABLE I

| Time | % |
|---|---|
| (Min) | Dissolution |
| 5 | 86 |
| 10 | 98 |
| 15 | 100 |
| 20 | 100 |
| 30 | 100 |
| 45 | 100 |
| 60 | 100 |
| 90 | 100 |

### EXAMPLE II

The following example demonstrates the enhanced dissolution of dimenhydrate and the effect of drug loading on matrix solubility.

Five grams of dimenhydrinate and 5 grams of Pluronic NF F68 were combined in a beaker and heated to approximately 90° C. until a clear liquid was obtained. The beaker was then removed from heat and the materials allowed to solidify slowly with no agitation. The resulting material, upon becoming completely solidified, was then ground to a powder and submitted for dissolution testing. The dissolution data is presented in TABLE II.

TABLE II

| Time | % |
|---|---|
| (Min) | Dissolution |
| 5 | 54 |
| 10 | 62 |
| 15 | 65 |
| 20 | 66 |
| 30 | 69 |
| 45 | 73 |
| 60 | 75 |
| 90 | 77 |

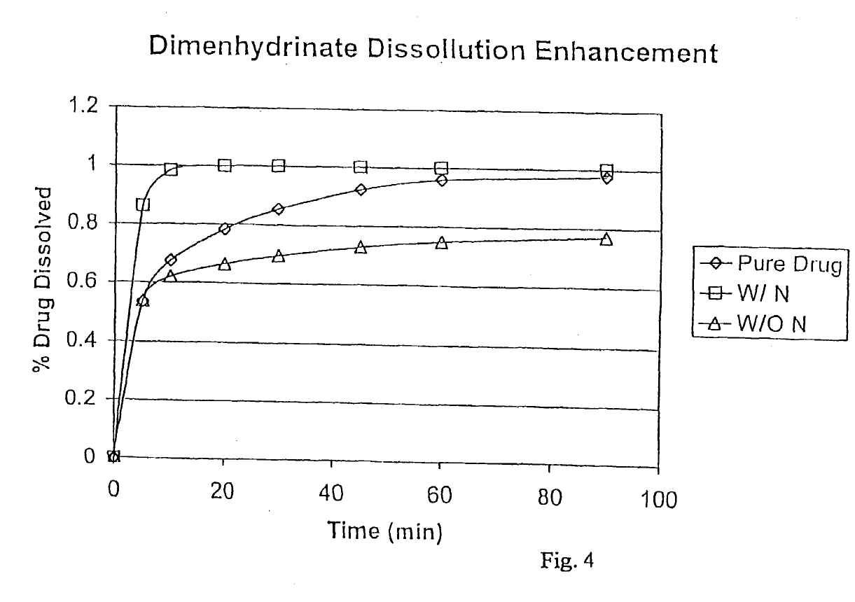

Dissolution data from Examples I and II are plotted in FIG. 4, with dissolution data for the unprocessed drug. The data shows that the drug/surfactant matrix was solidified under high shear, thus creating many nucleation sites, produced the smallest crystals and therefore the most rapid dissolution even poorer than the pure drug. In fact, the drug/surfactant matrix solidified under high shear exhibited a greater rate of dissolution than the pure drug. The material that was allowed to cool slowly grew large crystals and as a result exhibited a poor dissolution.

### EXAMPLE III

The following demonstrates the effect of intensive nucleation on matrix solubility and the effect of drug loading on matrix solubility.

Eight grams of dimenhydrinate and 2 grams of Pluronic NF F68 were combined in a beaker and heated to approximately 90° C. until a clear liquid was obtained. The material was then transferred to a mortar and pestle at room temperature and was allowed to solidify while being vigorously ground. The resulting material, upon becoming completely solidified, was then ground to a powder and submitted for dissolution testing. The dissolution data is presented in TABLE III.

TABLE III

| Time | % |
|---|---|
| (Min) | Dissolution |
| 5 | 46 |
| 10 | 70 |
| 15 | 82 |
| 20 | 87 |
| 30 | 95 |
| 45 | 101 |
| 60 | 103 |

### EXAMPLE IV

The following example demonstrates the effect of intensive nucleation on matrix solubility.

Five grams of dimenhydrinate and 5 grams of Pluronic NF F68 were combined in a beaker and heated to approximately 90° C. until a clear liquid was obtained. The beaker was then removed from heat and the materials were allowed to solidify slowly with no agitation. The resulting material, upon becoming completely solidified, was then ground to a powder and submitted for dissolution testing. Dissolution data is presented in TABLE IV.

TABLE IV

| Time | % |
|---|---|
| (Min) | Dissolution |
| 5 | 21 |
| 10 | 27 |
| 15 | 34 |
| 20 | 39 |
| 30 | 48 |
| 45 | 60 |
| 60 | 69 |
| 90 | 82 |

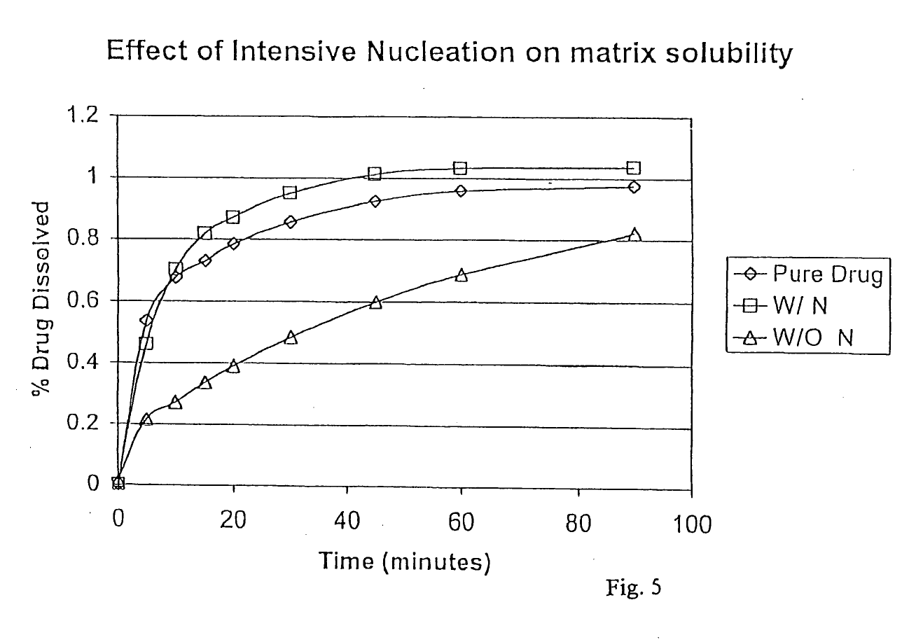

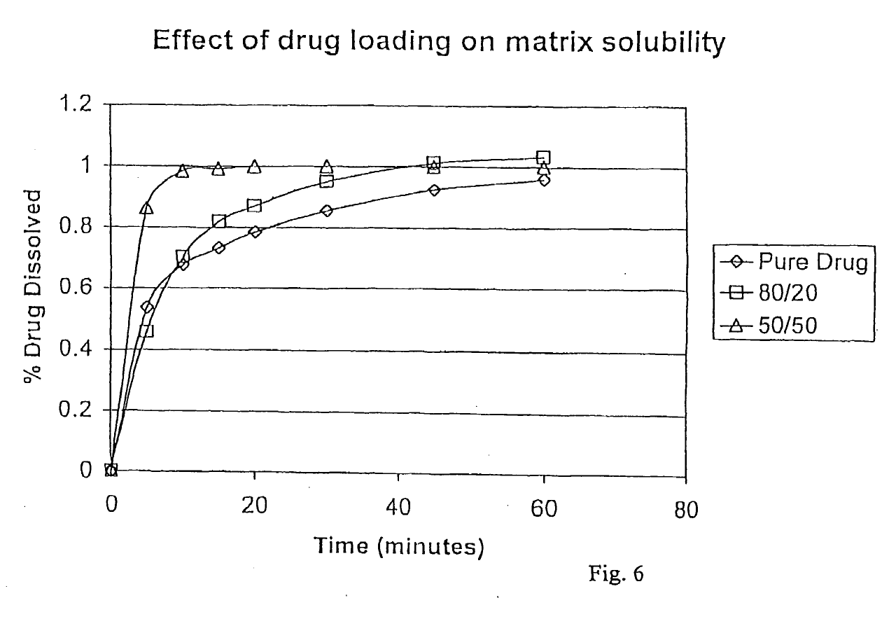

Dissolution data from Examples 3 and 4 are plotted in FIG. 5 with dissolution data for the unprocessed drug. Again, the data shows that the drug/surfactant matrix was solidified under high shear, thus creating many nucleation sites, produced the smallest crystals and therefore had the most rapid dissolution. Again, the drug/surfactant matrix solidified under high shear exhibited a greater rate of dissolution than the pure drug. The material that was allowed to cool slowly grew large crystals and exhibited poor dissolution as a result. Dissolution data from Examples 1 and 3 are plotted in FIG. 6 with dissolution data for the unprocessed drug. The data shows that the drug/surfactant ration of 50/50 exhibited the most rapid dissolution. The 80/20 matrix had a slower dissolution, probably due to the insufficient surfactant in the matrix to coat all of the drug particles completely.

The invention being thus described, it will be obvious that the same may be varied in many ways. Such variations are not to be regarded as a departure from the spirit or scope of the invention and all such modifications are intended to be included within the scope of the following claims.

1-17. (canceled)

18. A method of improving drug solubility by processing said drug into a surfactant-coated micro and nano-particulate form, the steps comprising:
(a) melting a sufficient amount of a drug in a molten surfactant miscible with said drug, to form a drug-surfactant mixture;
(b) heating the mixture to a temperature above said mixtures melting temperature, and below said drug's decomposition temperature until a clear mixture is formed; and
(c) cooling the mixture to approximately room temperature while continuously mixing under high shear in order to maximize precipitation of drug particles coated with said surfactant and having a greater rate of dissolution than pure unprocessed drug.

19. (canceled)

20. (canceled)

21. The method of claim 20, wherein said drug is micronized prior to adding to said surfactant.

22. The method of claim 18, wherein said drug is selected from the group consisting of: analgesics, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives, astringents, beta-adrenoceptor blocking agents, blood products and substitutes, cardiac inotropic agents, contrast media, corticosteroids, cough suppressants, diagnostic agents, diagnostic imaging agents, diuretics, dopaminergics, haemostatics, immunological agents, lipid regulating agents, muscle relaxants, parasympathomimetics, parathyroid calcitonin and biphosphonates, prostaglandins, radio-pharmaceuticals, sex hormones, anti-allergic agents, stimulants and anoretics, sympathomimetics, thyroid agents, vasodilators, xanthines, and mixtures thereof.

23. The method of claim 18, wherein said surfactant is an organic excipient.

24. The method of claim 23, wherein said excipient is selected from the group consisting of, gelatin, casein, gum acacia, cholesterol, tragacanth, stearic acid, benzalkonium chloride, calcium stearate, glyceryl monostearate, cetostearl alcohol, cetomacrogol emulsifying wax, sorbitan esters, polyoxyethylene alkyl ethers, macrogol ethers, polyoxyethylene castor oil derivatives, polyoxyethylene sorbitan fatty acid esters, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, and polyvinylpyrrolidone, polyvinyl alcohol, polyvinyl pyrrolidone, dextran, lecithin, polyvinylpyrrolidone, and organic solvent.

25. The method of claim 18, wherein said surfactant is nonionic or anionic.

26. The method of claim 18, wherein said drug is used in a pharmaceutically acceptable carrier.

27. The method of claim 26, wherein said pharmaceutically acceptable carrier is selected from the group consisting of diluents, binders, adhesives, lubricants, plasticizers, disintegrants, colorants, bulking substances, flavorings, sweeteners, buffers, and absorbents.

28. The method of claim 27, wherein said binder is selected from the group consisting of hydroxypropylmethylcellulose, ethylcellulose, povidone, acrylic, methacrylic acid co-polymers, pharmaceutical glaze, gums, and milk derivatives.

29. (canceled)

30. The method of claim 18, wherein said particles have a size of less than 5 microns.

31. The method of claim 18, wherein said particles have a size of less than 400 nm.

32. The method of claim 18, wherein said particles have a size of less than 250 nm.

33. The method of claim 18, wherein the average particle size is less than about 100 nm.

34. The method of claim 18, wherein the average particle size is less than about 250 nm.

35. The method of claim 18, wherein at least 50% of said particles have a size of less than 5 microns.
